(19) United States
(12) Patent Application Publication
(10) Pub. No.: US 2020/0346587 A1
(43) Pub. Date: Nov. 5, 2020

(54) DEODORIZING DEVICE AND SPREADER CLAMP FOR A DEODORIZING DEVICE

(71) Applicant: Supair-Tel AG, Glattbrugg (CH)

(72) Inventor: Rudolf Heinrich Guggenheim, Wollerau (CH)

(21) Appl. No.: 16/862,692

(22) Filed: Apr. 30, 2020

(30) Foreign Application Priority Data
May 2, 2019 (CH) 0586/19

(51) Int. Cl. B60R 11/00; B60H 3/00; A61L 9/12

(52) U.S. Cl. CPC B60R 11/00; B60H 3/0028; A61L 9/12; A61L 2209/15; B60R 2011/0071; B60R 2011/0059; B60R 2011/0008

(57) ABSTRACT

A deodorizing device for a vehicle comprises at least one deodorizing body and at least one spreader clamp fixed in or at the deodorizing body. The spreader clamp is intended for mounting the deodorizing body to a grid or to one or more slats/lamellae of an air outlet in the interior of a vehicle. The spreader clamp has two elastically deformable clamping members that extend in longitudinal direction of the spreader clamp and away from the deodorizing body and that are opposite one another transverse to the longitudinal direction. The clamping members are made of a first plastic material. Portions of the clamping members that are directly opposite one another, however, are of a second, softer plastic material than the first plastic material. This effects a good fixation at the air outlet and in addition prevents its scratching.

### CROSS REFERENCES TO RELATED APPLICATIONS

This application claims the priority of Swiss patent application 0586/19, filed May 2, 2019, the disclosure of which is incorporated herein by reference in its entirety.

### TECHNICAL FIELD

The invention regards a deodorizing device for a vehicle comprising at least one deodorizing body and at least one spreader clamp attached to or in the deodorizing body. Said spreader clamp is intended for mounting the deodorizing body on a grid or on one or more slats/lamellae of an air outlet in the interior of the vehicle. For enabling such mounting, the spreader clamp has two elastically deformable clamping members extending in longitudinal direction of the spreader clamp and arranged opposite one another transversely to the longitudinal direction. Said clamping members are formed of a first plastic material. In addition, the invention also regards a spreader clamp for the mentioned deodorizing device.

### BACKGROUND ART

Deodorizing devices comprising a deodorizing body and at least one means fixed to or within the deodorizing body, wherein said means is intended for mounting the deodorizing body on a grid or on one or more slats/lamellae of an air outlet in the interior of a vehicle, are known. The deodorizing body is a deodorant comprising body of any shape. Such deodorizing devices are e.g. known from WO 2017/108708 A1, WO 2015/179992 A1 and WO 2012/129709 A1. In particular, the means for mounting the deodorizing body on a grid or on slats/lamellae can be a spreader clamp that elastically expands upon insertion into the air outlet when pushed into grid openings or between slats/lamellae and then holds the deodorizing body on the grid or the slats/lamellae due to the restoring force of the elastically expanded clamp. Such a clamp enables the easy removal of the deodorizing device, once the deodorants are used up and the deodorizing device has to be replaced by a new one.

### DISCLOSURE OF THE INVENTION

Hence, it is a general object of the invention to provide an improved deodorizing device that enables easier mounting and avoids damages of the vehicle.

Now, in order to implement these and still further objects of the invention, which will become more readily apparent as the description proceeds, the deodorizing device for a vehicle is manifested by the features that said deodorizing device comprises at least one deodorizing body and at least one spreader clamp fixed in or at the deodorizing body, said spreader clamp is intended for mounting the deodorizing body to a grid or one or more slats/lamellae of an air outlet in the interior of the vehicle, said spreader clamp has two elastically deformable clamping members extending in a longitudinal direction of the spreader clamp and away from the deodorizing body, said clamping members are opposite one another transversely to the longitudinal direction, wherein said clamping members are formed of a first plastic material and wherein directly opposite portions of the clamping members are of a second, softer plastic material than the first plastic material.

On the one hand this enables a better adhesion of the spreader clamp at the grid or the slats/lamellae in that the softer plastic enables a form fit with the form of the grid or the slats/lamellae since the softer plastic material is to some extent deformable when subjected to the clamping force of the spreader clamp. In addition, the softer plastic material avoids damages of the grid or the slats/lamellae, respectively, in particular a scratching of these parts since as a rule these parts are manufactured from a harder plastic material than the softer plastic material of the spreader clamp. In the deodorizing device of the present invention, the deodorizing body can be any deodorizing body as used in vehicles and known to the skilled person.

In a preferred embodiment, the first plastic material is a thermoplastic material, in particular acrylonitrile butadiene styrene (ABS) and the second plastic material is a thermoplastic elastomer or a thermoplastic rubber elastic plastic material. With such a combination of plastic materials, the spreader clamp of the deodorizing device can be produced in one step by means of multi component injection molding and thus in a cost-effective manner.

It is also preferred that at least one of the clamping members has a clamping part that is bent back in direction of the deodorizing body. On this clamping part a portion of the second plastic material is provided. This allows to en-hance the clamping force or to obtain a good fixation at e.g. thin slats/lamellae, respectively. The bent back clamping part in combination with the softer plastic material at the bent back clamping part provides an especially good fixing and also an especially good prevention of damages or scratches, respectively, of the ventilation elements of the vehicle is obtained.

It is further preferred that the spreader clamp is provided with a fixing section designed for being fixed in the deodorizing body. Said fixing section comprises at least one retaining element and a support plate for the deodorizing body. This allows a good fixation in the deodorizing body so that handling of the deodorizing device with the spreader clamp that clamps very well at the ventilation element of the vehicle does not bear the risk that the spreader clamp separates from the deodorizing body.

A further problem to be solved by the present invention is to provide an improved spreader clamp for a deodorizing device as indicated at the start.

This problem is solved by a spreader clamp for a deodorizing device as described above, which spreader clamp for mounting a deodorizing body to a grid or to slats/lamellae of an air outlet in the interior of a vehicle has two elastically deformable clamping members that extend in longitudinal direction of the spreader clamp and away from the deodorizing body and opposite one another transversely to the longitudinal direction, said clamping members being formed of a first plastic material, wherein directly opposite portions of the clamping members are formed of a second, softer plastic material than the first plastic material.

With this spreader clamp the same advantages are obtained as indicated above with regard to the deodorizing device.

### BRIEF DESCRIPTION OF THE DRAWINGS

The invention will be better understood and objects other than those set forth above will become apparent when consideration is given to the following de-tailed description thereof. Such description makes reference to the annexed drawings, wherein:

FIG. 1 is a sectional view of one embodiment of a deodorizing device of the present invention with an only partially and schematically shown deodorizing body; and

FIG. 2 is a graphical presentation of a clamp of the present invention.

### MODES FOR CARRYING OUT THE INVENTION

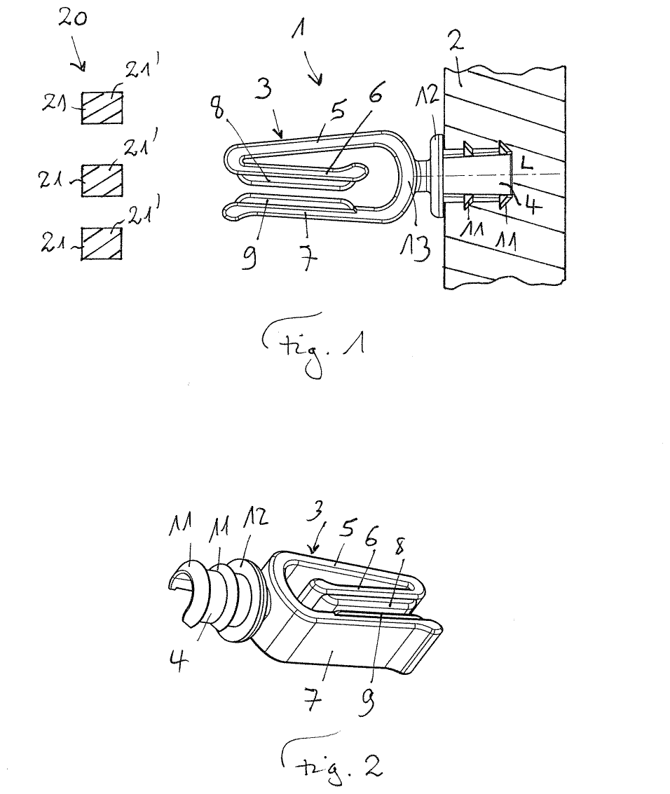

FIG. 1 shows a sectional view of a deodorizing device 1, comprising a deodorizing body 2 and a spreader clamp 3 fixed therein. FIG. 2 shows the spreader clamp 3 alone. The deodorizing body 2 can be any deodorizing body as used in cars that is a plastic body comprising deodorants, a body made of a po-rous material and soaked with deodorants or a container containing liquid deodorants that are by and by released through openings in the container. In the vehicle, the deodorizing body is mounted to an only hinted air outlet 20 of the vehicle by the spreader clamp 3. The air outlet has a grid 21 or slats/lamellae 21′ as this is known by the skilled person and only hinted in FIG. 1. When the spreader clamp 3 is inserted into the grid or between the slats/lamellae with its clamping members, the elastically deformable elastic clamping members spread out and hold on the grid or slats/lamellae by their restoring force so that the deodorizing body 2 or the whole deodorizing device 1, respectively, are mounted on the vehicle. In use, the deodorizing body is streamed around by the air leaving the ventilation of the vehicle. If the deodorant in the deodorizing body is used up, the spreader clamp can be pulled off the grid or slats/lamellae. In the example shown, the spreader clamp of the deodorizing device is fixed in the deodorizing body by a fixing section 4. As an alternative, the spreader clamp can be fixed at the outside of the deodorizing body.

The spreader clamp has a longitudinal direction indicated in FIG. 1 by means of a broken longitudinal axes L. The clamping members 5 and 7 extend in longitudinal direction and away from the deodorizing body 2 or the fixing section 4, respectively, so that they are suitable for being inserted into the ventilation opening of the vehicle. Viewed in direction transverse to the longitudinal direction, the clamping members are opposite one another so that they form a clamp or a clip. In the present context, these terms are understood by the skilled person as having the same meaning and are self-explanatory with regard to the Figures and the ex-plained function.

In FIG. 1 a preferred embodiment is shown, wherein the spreader clamp 3 of the deodorizing device 1 has a clamping member 5, said clamping member 5 having a clamping part 6 that is bent back in the direction of the deodorizing body 2 or in direction of the fixing section 4 of the spreader clamp 3. The other clamping member 7 is not identically embodied and extends in longitudinal direction in simple form, i.e. without being bent back. In another embodiment, the clamping member 5 could be designed like clamping member 7 or without a clamping part that is bent back, respectively. In yet another embodiment both clamping members 5 and 7 can be provided with a part that is bent back. In the example shown both clamping members 5, 7 are together arranged at the fixing section 4 or connected in front of the fixing section in an area 13, respectively. The clamping members 5, 7 can also extend from the mounting section 4 sepa-rately.

The elastically deformable clamping members 5, 7 being opposite to one another transversely to the longitudinal direction are formed from a first plastic material, while the directly opposite portions 8, 9 of the clamping members 5 (with the clamping part 6) and 7 are formed from a second, softer plastic material than the first plastic material. In a preferred embodiment, the fixing section 4 of the spreader clamp 3 is formed of the first plastic material. The first and the second plastic material preferably are thermoplastic materials enabling the production of the spreader clamp 3 by means of multi component injection molding. Acrylonitrile butadiene styrene (ABS) is a preferred first plastic material and the second plastic material is a thermoplastic elastomer that is a rubber-elastic plastic that is softer than the first plastic material. The fixing section 4 that is designed for being fixed in the deodorizing body 2 and that is also formed of the first plastic material, preferably has at least one retaining element 11 that provides good adhesion of the spreader clamp 3 of the deodorizing device in the deodorizing body 2 of the deodorizing device. In another preferred embodiment, a support plate 12 for supporting the exterior of the deodorizing body 2 is also provided at the spreader clamp 3. Usually, the spreader clamp 3 is a solid body made of the two plastics with a hol-low cylindrical fixing section 4 and the illustrations in FIG. 1 and FIG. 2 are to be understood this way.

While there are shown and described presently preferred embodiments of the invention, it is to be distinctly understood that the invention is not limited thereto but may be otherwise variously embodied and practiced within the scope of the following claims.

1. A deodorizing device for a vehicle, said deodorizing device comprising at least one deodorizing body and at least one spreader clamp fixed in or at the deodorizing body, said spreader clamp being intended for mounting the deodorizing body to a grid or one or more slats/lamellae of an air outlet in the interior of the vehicle, said spreader clamp having two elastically deformable clamping members extending in a longitudinal direction of the spreader clamp and away from the deodorizing body, said clamping members being opposite one another transversely to the longitudinal direction, wherein said clamping members are formed of a first plastic material and wherein directly opposite portions of the clamping members are of a second, softer plastic material than the first plastic material.

2. The deodorizing device of claim 1, wherein the first plastic material is a thermoplastic material and the second plastic material is a thermoplastic elastomer.

3. The deodorizing device of claim 2, wherein the first plastic material is acrylonitrile butadiene styrene (ABS).

4. The deodorizing device of claim 1, wherein at least one of the clamping members has a clamping part that is bent back in direction to the deodorizing body and wherein said clamping part is provided with one of the portions made of the second plastic material.

5. The deodorizing device of claim 1, wherein the spreader clamp has a fixing section designed for being fixed within the deodorizing body, which fixing section is also formed of the first plastic material.

6. The deodorizing device of claim 5, wherein the fixing section is provided with at least one retaining element and with a support plate for supporting the exterior of the deodorizing body.

7. A spreader clamp for a deodorizing device of claim 1, said spreader clamp being intended for mounting a deodorizing body to a grid or to one or more slats/lamellae of an air outlet in the interior of a vehicle, said spreader clamp having two elastically deformable clamping members that extend in longitudinal direction of the spreader clamp and away from the deodorizing body, said clamping members being opposite one another traverse to the longitudinal direction, wherein said clamping members are formed of a first plastic material, and wherein directly opposite portions of the clamping members are formed from a second, softer plastic material than the first plastic material.

8. The spreader clamp of claim 7, wherein the first plastic material is a thermoplastic material and the second plastic material is a thermoplastic elastomer.

9. The spreader clamp of claim 8, wherein the first plastic material is acrylonitrile butadiene styrene (ABS).

10. The spreader clamp of claim 7, wherein the spreader clamp has a fixing section designed for being fixed within the deodorizing body, said fixing section being provided with at least one retaining element and with a support plate for supporting the exterior of the deodorizing body.

11. The spreader clamp of claim 10, wherein at least one of the clamping members has a clamping part that is bent back in direction of the fixing section and wherein said clamping part is provided with one of the portions made of the second plastic material.
